(19) United States
(12) Patent Application Publication
(10) Pub. No.: US 2008/0206300 A1
(43) Pub. Date: Aug. 28, 2008

(54) Shaped Article

(76) Inventors: Marc Bohner, Grenchen (CH); Anna Malsy, Lyss (CH)

Correspondence Address: SCHWEGMAN, LUNDBERG & WOESSNER, P.A., P.O. BOX 2938, MINNEAPOLIS, MN, 55402, US

(21) Appl. No.: 11/916,693

(22) PCT Filed: Jun. 9, 2005
(86) PCT No.: PCT/CH05/00320

§ 371 Date: Dec. 6, 2007

(51) Int. Cl. A61F 2/28; A61K 38/19; A61P 19/00; B01D 15/08

(52) U.S. Cl. 424/423; 514/12; 210/656

(57) ABSTRACT

The shaped article is obtained via a cementitious reaction of a particulate composition reactive with water, whereby said reaction is obtained between said composition and an aqueous, liquid or gaseous phase. The particles of the shaped article are present in the form of interlocked particles, whereby the interlocking of said particles is obtained in a 100% water-saturated atmosphere.

The invention relates to a shaped article according to the preamble of claim 1.

Shaped articles made up of calcium phosphate materials are known to be osteoconductive bone substitutes, i.e. bone forms in the bone substitute when the bone substitute is in close apposition to bone. A long time ago it was further suggested that coral-derived apatites could also be osteoinductive, i.e. bone can form within the bone substitute even though the bone is in bone ectopic site. Since then, there has been numerous studies showing that apatites and calcium phosphate materials can be osteoinductive. Nevertheless, there is so far no clear understanding for this phenomenon. Factors such as calcium phosphate chemistry, porosity, pore size, pore shape, implant location (e.g. intramuscular or subcutaneous, back or thigh), implant type (e.g. granule or block), pre-hardened or injected cement, block shape, implantation time, and animal type have been tested. Generally, more bone has been found (i) at longer implantation times, (ii) in less resorbable calcium phosphates, (iii) in baboons, dogs and pigs (rather than rabbits, mice and rats), (iv) in more microporous materials, (v) in macropores, in particular macropore concavities, (vi) in blocks (rather than granules), and (vii) intramuscularly (rather than subcutaneously).

Until now, most efforts made on the material side have been focused on the effect of composition, micro- and macroarchitecture. Little has been done to assess the effect of nanoarchitecture despite the fact that bone does only contain calcium phosphate nanoparticles rather than microparticles.

The invention intends to provide a shaped article having a higher specific surface area. It is based on new architectures of bone substitutes that strongly enhance their osteoinductivity (via an increase of protein adsorption). These new architectures can be obtained with a number of calcium phosphate cement compositions.

Until now bone substitute in a granular or block form are obtained by traditional ceramic processing methods, i.e. in particular by sintering the ceramic at high temperature in order to strengthen the material. Sintering has the great disadvantage that the initially large surface area of the bone substitute is substantially reduced during the process. Typically, the specific surface area (SSA) of sintered materials is close to 0.1-1.0 m²/g whereas initial specific surface areas can easily reach 100 m²/g. This is the case for the material described in the US patent of Ying et al (U.S. Pat. No. 6,013,592) who discloses an agglomerated compound which is made up of spherical particles obtained by crystallization from a solvent and which are pressed or sintered to form the shaped article. In the absence of sintering, the (pressed) shaped article has almost the same specific surface area as the powder used to obtain the shaped article, but no mechanical stability. With sintering, the shaped article has a much larger mechanical stability but a drastically lower specific surface area, typically lower than 10 to 20 m²/g.

The invention solves the posed problem with a shaped article that displays the features of claim 1.

The shaped articles according to the invention are obtained via a cementitious reaction between an aqueous phase (gas or liquid) and reactive compounds. The particles formed during cement curing reaction grow until particle interlocking occurs. As a result, the shaped article does not need pressing or sintering (as in Ying et al) to achieve a high mechanical stability. Additionally, any shape can be obtained since the cement paste can be injected into any geometrical form and does not shrink during setting (sintering as promoted by Ying et al is associated with shrinkage). Finally, when suitable additives (as e.g. so-called “growth inhibitors”, which are described in more detail below) are used, the specific surface area (SSA) of the shaped article becomes very large, much larger than the values typically obtained by other methods. Values above 100 m²/g can be reached.

The specific surface area (SSA) of the shaped article according to the invention is not the only important parameter determining protein adsorption. As proteins have a certain size, the shaped article should preferably have nanopores big enough for proteins to penetrate the structure. Nanopores result from the gaps between interlocked particles. Nanopores larger than 10 nm are of great interest because most proteins can then penetrate the structure.

Said cementitious reaction is preferably obtained by incubation of said composition in a closed atmosphere that has a 100% relative humidity or that can be saturated by water present in the composition to reach 100% relative humidity. The incubation in a saturated atmosphere has the advantage that it allows the obtaining of blocks without disintegration and good control of the interlocked structure. In another embodiment the composition may contain water.

In a further embodiment the particles are made of crystallites. Crystallites are coherent (free of defects) crystal units that diffract in phase. The crystallite size is a measurement of adjacent, repeating crystalline units. The term crystallite size is commonly substituted for the term grain size when related to metallic films. The crystallite size is only equivalent to the grain size if the individual grains are perfect single crystals free of defects, grain boundaries, or stacking faults. The crystallite size of the shaped article is of importance because the solubility of a given compound depends on it: the smaller the size is, the more soluble the compound is.

As apatite compounds tend to be resorbed too slowly, it is advantageous to have a crystallite size as small as possible. So, apatite crystallites should have a size (measured by X-ray diffraction) typically smaller than 20 nm and preferably smaller than 15 nm.

Calcium phosphate cements have been known for two decades already. Calcium phosphate cements basically consist of one or several calcium phosphate powders and an aqueous solution. The calcium phosphate powder(s) dissolve(s) in the aqueous solution and a new calcium phosphate phase precipitates. Traditionally, cements have been used as injectable or moldable bone substitute and not for the synthesis of granules and blocks. As a result, authors have not focused their attention to the effects of cement chemistry on the cement nanostructure, but rather on the mechanical properties. Features such as particle size, specific surface area, or nanopore size distribution have not been measured and hence optimized. Moreover, the synthesis of nanostructured granules and blocks set other requirements for production than cements as will be shown in the next lines.

In fact, the easiest method to increase the specific surface area (SSA) of the shaped article is to synthesize it in the presence of so-called “growth inhibitors”.

These chemical compounds prevent the growth of particles, hence resulting in numerous nanoparticles. As growth inhibitors strongly slow down the curing/setting reaction of the cement, the cement does not harden within minutes as required for traditional clinical applications of cements but within days. Typically, cements prepared in a lab are incubated in an aqueous solution. Here, if the shaped articles are placed in an aqueous solution, the paste disintegrate. Disintegration prevents the obtention of a mechanically stable block. Moreover, the external cement surface in contact with the incubating solution has a different nanostructure than the bulk, so it is impossible to control the nanostructure of the block. If the shaped articles consisting of uncured cement paste are kept in air for several days (until setting occurs), the shaped articles dry and do not harden. Here, the problem was solved by incubating the shaped articles in a closed atmosphere. As cement pastes always contain an excess of water, the excess water evaporates into the closed atmosphere until 100% relative humidity is obtained (the ratio between cement volume and volume of closed atmosphere must be large enough to reach 100% relative humidity). Another problem is the very slow curing reaction in the presence of growth inhibitors. Here, the problem was solved by curing the cement at elevated temperature, typically higher than 37° C., e.g. 60-80° C. Higher temperatures (even higher than the boiling point of water, e.g. 120° C. or 250° C.) are also possible but tend to lead to the formation of much larger particles and hence reduce the specific surface area.

Various chemical compounds can be used to modify the nanostructure of the shaped articles. For sterilization purposes, it is advantageous to use inorganic additives. Most common examples are Mg, carbonate, or pyrophosphate ions. Organic additives could also be used. Peptides, proteins, citrate ions, and in general carboxylated compounds (COOH group) are potent additives.

For the synthesis of blocks, it might be of great interest to have macropores (size larger than 50 μm) in the shaped article in order to promote blood vessel ingrowth and hence faster bone formation and ceramic resorption. Such large pores can be obtained by combining the cement paste with another phase such as a solid, a liquid or a gas. The only conditions set to form macropores are that the solid, liquid or gas phase can be easily removed from the cement paste during or after hardening to leave empty macropores. Many techniques can be used, such as the use of ice or saccharides particles, the use of a hydrophobic liquid, or gas (foaming technique).

In a special embodiment the shaped article comprises inorganic particles, which are in a mechanically stable agglomerated state, e.g. calcium phosphate. In a further embodiment the particles are nanoparticles.

In a further embodiment particles are used which are not spherical. Preferably the particles have a needle-like or plate like form, which allows to obtain a higher specific surface area. In a further embodiment the shaped article is obtained by precipitation. Compared to pressing or sintering, precipitation allows to obtain a higher specific surface are.

The shaped article may also be obtained by crystallization in a gaseous phase at a temperature in the range of 0-250° C., preferably of 50-100° C. The crystallization may be effected under pressure during part or all of the crystallization process.

The specific surface area (SSA) of the agglomerated particles should preferably be superior to 40 m²/g. A larger specific surface area leads to more protein adsorption and hence a higher osteoinductivity. Therefore the specific surface area of the agglomerated particles is preferably superior to 50 m²/g, and typically superior to 80 m²/g.

The compressive strength of the shaped article is preferably superior to 1 MPa, and typically superior to 10 MPa.

The agglomerated particles should preferably have interconnected pores, resulting from the interstices between the single particles. Preferably 50 to 80% of said pores should be larger than 10 nanometer in diameter. Such a structure is open for the diffusion of proteins. The porosity should preferably be larger than 20% (typically larger than 40%) and preferably lower than 95% (typically lower 93%). Higher values would lead to an unacceptable brittleness of the material.

In a special embodiment the particles should preferably have an apatitic composition. The should preferably have a Ca/P molar ratio of 0.5 to 2.5, typically of 1.0 to 2.0.

The shaped article may advantageously be impregnated with an inorganic or organic substance that promotes or controls peptide and/or protein adsorption. The impregnation may be effected with a therapeutic agent, preferably for the musculoskeletal system or circulatory system. The therapeutic agent for the musculoskeletal system may be chosen from the group of the cytokines or drugs against osteoporosis. The therapeutic agent for the circulatory system may be a clotting preventing agent. Instead of impregnating the shaped article after said cementitious reaction has taken place the therapeutic agent may be included in said particulate composition already before said cementitious reaction takes place.

In a further embodiment the shaped article may contain macropores, preferably with a size larger than 50 micrometers in diameter. The macropores may be interconnected, preferably with an interconnection size larger than 50 micrometers.

The shaped article according to the invention may be used in the medical field as bone substitute but also in the non-medical field, e.g. for chromatography purposes, preferably in a chromatographic separation column.

The invention and additional configurations of the invention are explained in even more detail with reference to the following examples of manufacture and to the figures.

Shown are:

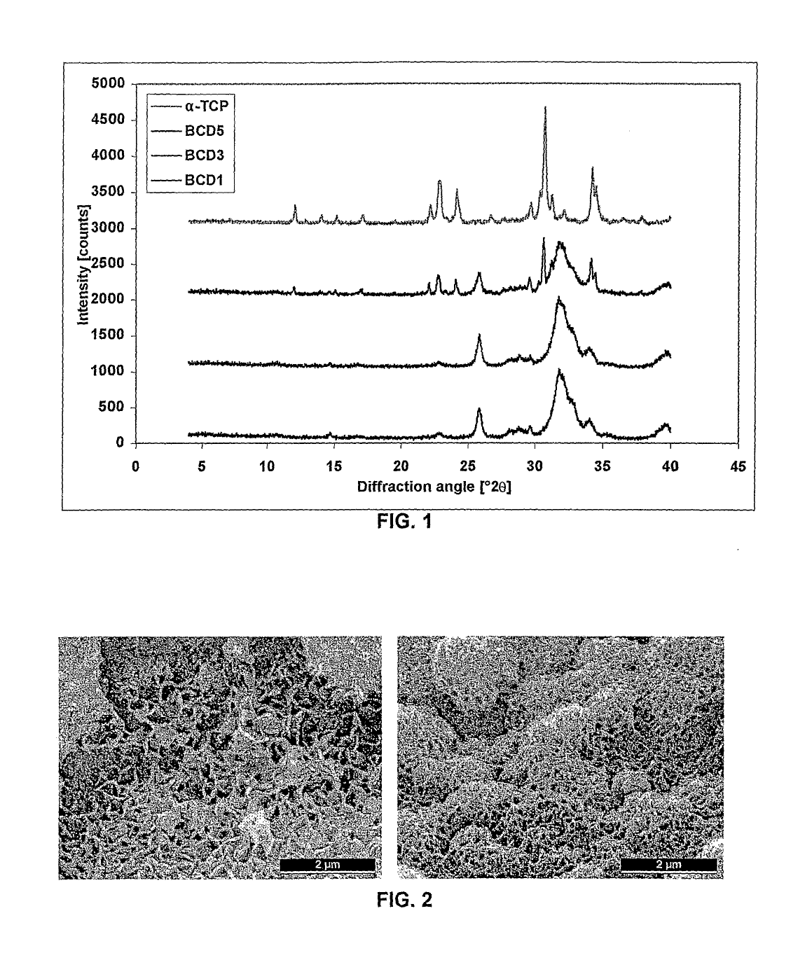

FIG. 1

XRD patterns of samples BCD1, BCD3, BCD5 and α-TCP (from bottom to top, respectively). Synthesis conditions: 60° C., 3 days; and

FIG. 2

Microstructure of samples BCD1 (left) and BCD5 (right) as observed by SEM (magnification 20000×).

### EXAMPLE 1

The solid phase was a mixture of α-tricalcium phosphate (α-TCP), calcium sulfate dihydrate (CSD), calcium carbonate (CC), and magnesium hydrogen phosphate trihydrate (MgP). The liquid phase was a solution of sodium hydrogen phosphate 0.5 M with Ethanol 99.9%. The LIP ratio is 0.43 ml/g (Table 1).

TABLE 1
Composition of three samples BCD1, BCD3 and BCD5

| | a-TCP | CSD | MgP | CC | NaP | EtOH | dH₂O |
|---|---|---|---|---|---|---|---|
| Sample | [g] | [g] | [g] | [g] | [ml] | [ml] | [ml] |
| BCD1 | 3.63 | 0.37 | 0.019 | 0.060 | 0.702 | 0.035 | 1.017 |
| BCD3 | 3.63 | 0.37 | 0.057 | 0.180 | 0.728 | 0.109 | 0.985 |
| BCD5 | 3.63 | 0.37 | 0.095 | 0.300 | 0.756 | 0.189 | 0.945 |

Each cement (20 batches in total=80 g) was prepared under laminar flow conditions by adding the previously-mixed and sterilized powder to the ultrafiltrated liquid in a small autoclaved beaker. The paste was homogenized for 45 s with a spatula and introduced into a cylindrical form (previously autoclaved). The form was then introduced into 20 ml container (previously autoclaved), the container was closed with a lid, and incubated in an oven at 60° C. for 3 days. The cylinders were then dried under vacuum at 80° C. until constant weight was reached. Finally, the cylinders were ground and sieved, and the granule fraction of 0.7-1.4 mm was kept. The latter granules were extensively washed in ethanol to remove all dust particles resulting from grinding, dried in air at 60° C., and finally sterilized by gamma irradiation. One part of the granules was used for characterization and one part was implanted in vivo (See hereafter).

In FIG. 1 the X-ray diffraction (XRD) patterns for the three compositions BCD1, BCD3 and BCD5 are shown. Clearly, BCD1 and BCD3 have reacted and have been converted to calcium deficient hydroxyapatite (CDHA), whereas BCD5 has not completely reacted. There is no significant difference between the spectrum of BCD1 and BCD3. Both present intense reflexes in the range between 31.7° 2θ, and one slightly above 25.8° 2θ. These peaks are typical for apatite compounds. The comparison between these spectra and those of “standard” CDHA shows that BCD1 and BCD3 have a more amorphous structure as “standard” CDHA. This is characterized by a broadening and a shortening of the diffraction peaks. The crystallite size determined by the full width at half maximum peak intensity (FWHM) as described by the Scherrer equation is 18, 18, and 16 nm for BCD1, BCD3 and BCD5, respectively.

The comparison of BCD5 and α-TCP shows that BCD5 contains remnants of α-TCP, signifying that the hydrolysis of α-TCP in CDHA is not total during the incubation time, maybe because of the action of different added ions on the setting time.

In FIG. 2 the microstructure is observed by scanning electron microscopy (SEM). Only the pictures of BCD1 (on the left side) and BCD5 (on the right side) are presented here (magnification 20000×). BCD1 presents large platelets-like crystals with an organization in clusters which are visible at magnification 10000× (not represented here). BCD5 presents also a cluster organization with a smaller diameter than the one of BCD1 and the crystals are not more in platelet-like shape but in tubular shape with no sharp edges. Both structures have over 90% of the pores larger than 20 nm.

On Table 2 are reported the SSA measurements for α-TCP and three samples BCD1, BCD3 and BCD5.

TABLE 2
SSA of samples BCD1, BCD3 and BCD5. Results are expressed
as mean ± SD.

| Sample | SSA [m²/g] |
|---|---|
| α-TCP | 2.2 ± 0.2 |
| BCD1 | 35.2 ± 1.8 |
| BCD3 | 39.3 ± 2.3 |
| BCD5 | 69.0 ± 0.4 |

The adsorption of bovine serum albumin is 0.58, 0.57 and 0.55 mg/m² for BCD1, BCD3 and BCD5, hence resulting in 20.4, 22.4, and 38.0 mg BSA/g CDHA.

Granules of formulations BCD3 and BCD5 were compared with β-tricalcium phosphate (β-TCP; chronOS™) granules (<0.5 m²/g surface) in vivo. Before implantation in the back of SCID mice carriers were freshly loaded with 2×10⁵ expanded human MSC or left as received. Implantations were done as follows: under general i.p. anesthesia and after disinfection of the back of the mice, three subcutaneous pockets were bluntly created through a one centimeter incision at the back. Two similar scaffolds of each group (BCD3, BCD5, chronOS™) were inserted into each subcutaneous pocket. The wound was closed with single interrupted sutures. The animals were sacrificed and the biomaterial/cell constructs were harvested at 8 weeks. Deposits of osteoid at the margins of ceramic occurred, contained human cells, and appeared in 10/16 MSC/BCD3 composites, in 14/16 MSC/BCD5 composites and only 2/16 MSC/β-TCP composites. Similar but significantly lower results were obtained for ceramic alone: 7/16 (BCD3), 12/16 (BCD5) and 0/16 (chronOS™).

Therefore BCD3 and BCD5 demonstrate a much higher osteoinductivity than chronOS™.

### EXAMPLE 2

The solid phase was a mixture of α-TCP (8 g), CC (8 g), monocalcium phosphate monohydrate (0.8 g), d.i. water (7.21 mL) and D-mannitol particles (17 g, sieved in the range of 0.25 to 0.5 mm). The liquid phase consisted of 7.21 ml of deionized water. Each cement (20 batches in total=33.8 g×20=676 g) was prepared under laminar flow conditions by adding the previously-mixed and sterilized powder to the ultrafiltrated liquid in a small autoclaved beaker. The paste was homogenized for 45 s with a spatula and introduced into a 30 ml large cylindrical form (previously autoclaved). The form was then placed into 100 ml container (previously autoclaved), the container was closed with a lid, and incubated in an oven at 90° C. for 1 day. Later, 50 ml of deionized water were added into the 100 ml container and incubated for one additional day at 90° C. (to dissolve mannitol particles and hence pores in the cement structure. Afterwards, the liquid was poured out and cylinders were dried under vacuum at 80° C. until constant weight was reached, and finally sterilized by gamma irradiation. The specific surface area of the resulting block was 45 m²/g. The crystallite size was 12 nm.

The compressive strength of the block after mannitol dissolution was 2.5 MPa whereas the total porosity was 76 vol %.

### EXAMPLE 3

The solid phase was a mixture of α-TCP (4 g), CC (1 g), and 0.1 g disodium dihydrogen pyrophosphate (Na₂H₂P₂O₇). The powders were mixed end-over-end for one hour (Turbula mixer), and pressed into a cylinder (diameter 10 mm; length: 3.8 cm (60% apparent density). The cylinder was then placed into a 100% relative humidity atmosphere at 125° C. for 6 hours. Drying was performed at the same temperature but in dry conditions. The cylinders were sterilized by gamma irradiation The specific surface area was 86 m²/g for a compressive strength of 65 MPa. The nanopore average size was 90 nm with 99% larger than 10 nm.

### EXAMPLE 4

6.67 g β-tricalcium phosphate powder was mixed with 3.33 g monocalcium phosphate monohydrate powder and 2.00 calcium sulfate hemihydrate powder. The liquid phase consisted of 4 ml deionized water. The cement was prepared under laminar flow conditions by adding the previously-mixed and gamma-sterilized powder to the ultrafiltrated liquid in a small autoclaved beaker. The paste was homogenized for 45 s with a spatula (sterile) and introduced into a cylindrical form (previously autoclaved). The form was then placed into 20 ml container (previously autoclaved), the container was closed with a lid, and incubated in an oven at 50° C. for three days. Afterwards, 5 ml deionized water (sterile) were added to the sample, and incubated for one more day at 50° C. Later, the liquid was removed, the cylinders were dried under vacuum at 50° C. until constant weight was reached, and finally sterilized by gamma irradiation. The specific surface area of the resulting block was 28.2 m²/g with a crystallite size of 25 nm.

1. A shaped article obtained via a cementitious reaction of a particulate composition reactive with water, whereby said reaction is obtained between said composition and an aqueous, liquid or gaseous phase;
characterized in that
A) said particles of said shaped article are present in the form of interlocked particles; whereby the interlocking of said particles is obtained in a 100% water-saturated atmosphere; and
B) the agglomerate formed by said particles has interconnected pores, resulting from the interstices between said single particles.

2. A shaped article according to claim 1, characterized in that it comprises inorganic particles, which are in a mechanically stable agglomerated state.

3. A shaped article according to claim 1, characterized in that said cementitious reaction is obtained by incubation of said composition in a closed atmosphere that has a 100% relative humidity or that can be saturated by water present in the composition to reach 100% relative humidity.

4. A shaped article according to claim 1, characterized in that said composition comprises a composition based on a calcium phosphate.

5. Article according to claim 1, characterized in that said composition contains water.

6. Article according to claim 1, characterized in that said particles are made of crystallites.

7. Article according to claim 1, characterized in that said particles are nanoparticles.

8. Article according to claim 6, characterized in that said crystallites have a size which is smaller than 20 nm, preferably smaller than 15 nm.

9. Article according to claim 1, characterized in that said particles are not spherical and preferably have a needle-like or plate like form.

10. Article according to claim 1, characterized in that it is obtained by precipitation.

11. Article according to claim 1, characterized in that it is obtained by crystallization in a gaseous phase at a temperature in the range of 0-250° C., preferably of 50-100° C.

12. Article according to claim 11, characterized in that said crystallization is effected under pressure during part or all of the crystallization process.

13. Article according to claim 1, characterized in that the specific surface area (SSA) of the agglomerated particles is superior to 40 m²/g.

14. Article according to claim 13, characterized in that said specific surface area of the agglomerated particles is superior to 50 m²/g, preferably superior to 80 m²/g.

15. Article according to claim 1, characterized in that it has a compressive strength superior to 1 MPa.

16. Article according to claim 15, it has a compressive strength superior to 10 MPa.

17. Article according to claim 16, characterized in that 50 to 80% of said pores are larger than 10 nanometer in diameter.

18. Article according to claim 1, characterized in that it has a porosity larger than 20%, preferably larger than 40%.

19. Article according to claim 1, characterized in that it has a porosity of less than 95%, preferably less than 93%.

20. Article according to claim 1, characterized in that said particles have an apatitic composition.

21. Article according to claim 1, characterized in that said particles have a Ca/P molar ratio of 0.5 to 2.5, preferably of 1.0 to 2.0.

22. Article according to claim 1, characterized in that it is impregnated with an inorganic or organic substance that promotes or controls peptide and/or protein adsorption.

23. Article according to claim 1, characterized in that it is impregnated with a therapeutic agent, preferably for the musculoskeletal system or circulatory system.

24. Article according to claim 23, characterized in that said therapeutic agent for the musculoskeletal system is chosen from the group of the cytokines or drugs against osteoporosis.

25. Article according to claim 23, characterized in that said therapeutic agent for the circulatory system is a clotting preventing agent.

26. Article according to claim 1, characterized in that a therapeutic agent is included in said particulate composition before said cementitious reaction takes place.

27. Article according to claim 1, characterized in that it contains macropores, preferably with a size larger than 50 micrometers in diameter.

28. Article according to claim 27, characterized in that said macropores are interconnected, preferably with an interconnection size larger than 50 micrometers.

29. Method of manufacture of a shaped article via a cementitious reaction of a particulate composition reactive with water characterized in that said cementitious reaction is obtained by incubation of said composition in a closed atmosphere that has a 100% relative humidity or that can be saturated by water present in the composition to reach 100% relative humidity.

30. Use of the article according to claim 1 a bone substitute or as a bone defect filler, preferably as an osteoinductive bone substitute or bone defect filler.

31. Use of the article according to claim 1 for chromatography purposes, preferably in a chromatographic separation column.
